(19) United States
(12) Patent Application Publication
(10) Pub. No.: US 2008/0319205 A1
(43) Pub. Date: Dec. 25, 2008

(54) Process for preparing 5-bromo-3-[(R)-1-methyl-pyrrolidin-2-ylmethyl]-1H-indole

(76) Inventors: Roman Bednar, Radun (CZ); Ondrej Simo, Trnava (SK); Pavel Blatny, Opava (CZ)

Correspondence Address: KENYON & KENYON LLP, ONE BROADWAY, NEW YORK, NY, 10004, US

(21) Appl. No.: 12/156,239

(22) Filed: May 29, 2008

Related U.S. Application Data
(60) Provisional application No. 60/932,206, filed on May 29, 2007.
(60) Provisional application No. 60/955,981, filed on Aug. 15, 2007.

(51) Int. Cl. C07D 209/08

(52) U.S. Cl. 548/468

(57) ABSTRACT

The invention encompasses a process for preparing 5-bromo-3-[(R)-1-methyl-pyrrolidin-2-ylmethyl]-1H-indole comprising reacting (R)-2-(5-bromo-1H-indole-3-carbonyl)-pyrrolidine-1-carboxylic acid benzyl ester with a reducing agent selected from the group consisting of sodium dihydro-bis(2-methoxyethoxy)aluminate, lithium tris[(3-ethyl-3-pentyl)oxy]aluminohydride, lithium tri-tert-butoxyaluminum hydride and diisobutylaluminium hydride. 5-bromo-3-[(R)-1-methyl-pyrrolidin-2-ylmethyl]-1H-indole is a key intermediate for preparing eletriptan and its salts thereof.

### CROSS-REFERENCE TO RELATED APPLICATIONS

This application claims the benefit of U.S. provisional application Ser. Nos. 60/932,206, filed May 29, 2007; and 60/995,981, filed Aug. 15, 2007, hereby incorporated by reference.

### FIELD OF THE INVENTION

The invention encompasses processes for the preparation of 5-bromo-3-[(R)-1-methyl-pyrrolidin-2-ylmethyl]-1H-indole (“BIP”), which is a key intermediate of eletriptan and salts thereof.

### BACKGROUND OF THE INVENTION

Eletriptan (“ELT”), 3-[[(R]-1-methyl-2-pyrrolidinyl)methyl]-5-[2-(phenylsulfonyl)ethyl]indole, having the following formula;

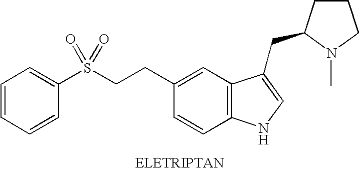

is used for the acute treatment of migraine with or without aura in adults. Eletriptan is a selective 5-hydroxytryptamine 1B/1D receptor agonist, which is administrated as eletriptan hydrobromide. Eletriptan tablets are marketed by Pfizer under the name RELPAX®.

Eletriptan and intermediates thereof, including 5-bromo-3-[(R)-1-methyl-pyrrolidin-2-ylmethyl]-1H-indole (“BIP”) are described in U.S. Pat. No. 5,545,644. Also disclosed is the synthesis of ELT, which is illustrated by the following scheme:

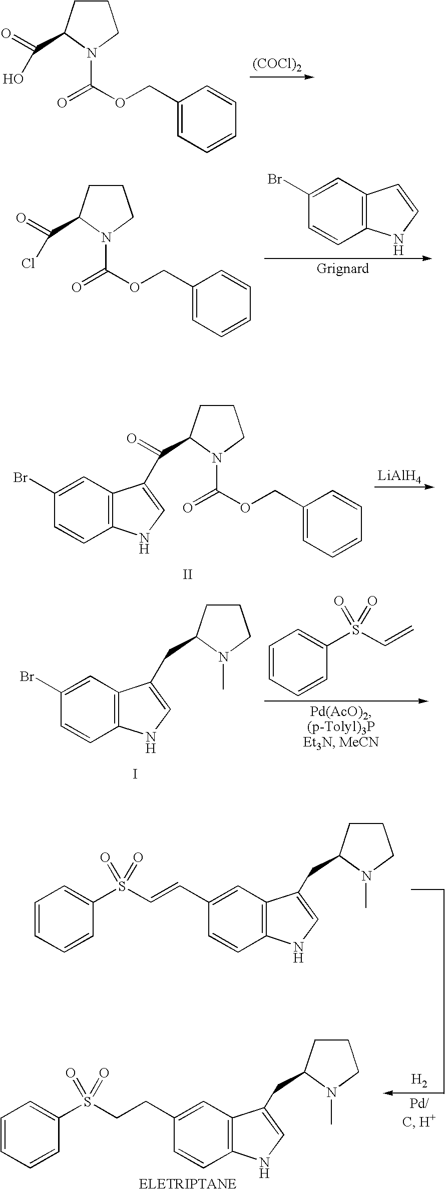

In the described process, intermediate I, BIP, is obtained by reacting intermediate II with lithium aluminium hydride (“LAH”). LAH spontaneously reacts with water, including atmospheric humidity, and the pure material is pyrophoric. The LAH is known as very unstable, and air-exposed samples are almost always contaminated with aluminium metal and or a mixture of lithium hydroxide and aluminium hydroxide, thus affecting the reactivity of the LAH powder. This leads to the use of a large excess of reagent in order to obtain moderate conversion.

Furthermore, the described process requires heating to reflux for a long period of time (39 hours in total, according to example 29 in U.S. Pat. No. 5,545,644) followed by a time consuming recovery process. The recovery process consists of diluting of the reaction mixture with ethyl acetate, filtering through cellulose filtration bar, as described in U.S. Pat. No. 5,545,644 example 27, and purifying the obtained oily like residue by silica gel chromatography, wherein, dichloromethane, ethanol and concentrated aqueous ammonia are used as a mobile phase. This process provides BIP, which is then converted to ELT.

Hence, there is a need in the art for an additional process for preparing BIP, which provides BIP in high yields and purity via a simple recovery process.

### SUMMARY OF THE INVENTION

In one embodiment, the present invention provides a process for the preparation of 5-bromo-3-[(R)-1-methyl-pyrrolidin-2-ylmethyl]-1H-indole (“BIP”), having the following formula

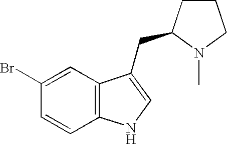

comprising: reacting (R)-2-(5-bromo-1H-indole-3-carbonyl)-pyrrolidine-1-carboxylic acid benzyl ester of formula II,

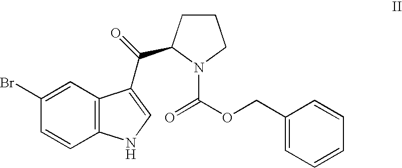

with a reducing agent selected from a group consisting of: sodium dihydro-bis(2-methoxyethoxy)aluminate (“SDMA”), Lithium tris[(3-ethyl-3-pentyl)oxy]aluminohydride, Lithium tri-tert-butoxyaluminum hydride (“TBLAH”) and Diisobutylaluminium hydride (“DIBALH”).

In another embodiment, the present invention provides a process for the preparation of eletriptan HBr comprising preparing BIP according to the process of the present invention, and converting it to eletriptan and salts thereof, preferably, to HBr salt.

### DETAILED DESCRIPTION OF THE INVENTION

The present invention relates to a process for preparing BIP of the following formula,

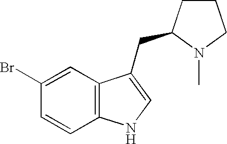

which is a key intermediate in the synthesis of ELT and salts thereof, especially, ELT-HBr.

The process of the present invention uses reducing agents such as sodium dihydro-bis(2-methoxyethoxy)aluminate (“SDMA”), Lithium tris[(3-ethyl-3-pentyl)oxy]aluminohydride, Lithium tri-tert-butoxyaluminum hydride (“TBLAH”) and Diisobutylaluminium hydride (“DIBALH”). These reducing agents are easier to handle compared to LAH because they are available in a solution rather than in a suspension in oil, as is the case for LAH. This also simplifies the recovery of BIP (exemplified in example 9 vs. the other examples).

In the prior art, LAH is constantly added to the reaction mixture in order to improve the conversion of OH-BIP, an intermediate of the reaction (for formula see infra), to BIP. However, although 3.9 equivalents of LAH are added to the reaction mixture, the conversion is not complete and the crude product is contaminated by OH-BIP. This impurity is difficult to remove and therefore can contaminate also the next intermediate.

Thus, these reducing agents, although considered to be less reactive than LAH, still provide high quality crude BIP in high yield (see example 5 vs. example 9), that can be purified easily by crystallization instead of column chromatography, as done in the prior art.

In one embodiment, the present invention encompasses a process for preparing BIP comprising: reacting (R)-2-(5-bromo-1H-indole-3-carbonyl)-pyrrolidine-1-carboxylic acid benzyl ester of formula II with a reducing agent selected from the group consisting of: SDMA, Lithium tris[(3-ethyl-3-pentyl)oxy]aluminohydride, TBLAH and DIBALH.

The process can be described by the following scheme:

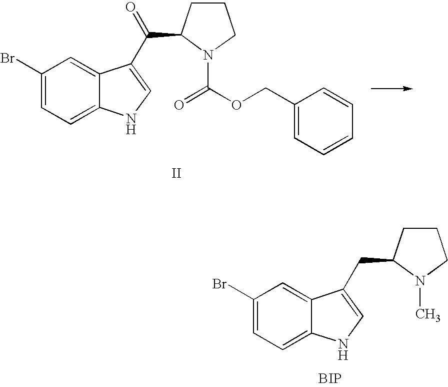

(R)-2-(5-bromo-1H-indole-3-carbonyl)-pyrrolidine-1-carboxylic acid benzyl ester of formula II can be prepared according to any method known in the art, e.g., a Grignard reaction as described in U.S. Pat. No. 5,545,644.

Prior to reacting with a reducing agent, (R)-2-(5-bromo-1H-indole-3-carbonyl)-pyrrolidine-1-carboxylic acid benzyl ester of formula II is combined with an organic solvent to provide a solution or a suspension, depending on the solvent used. Typically, suitable organic solvents for such a reaction are aprotic organic solvents.

Usually, the aprotic organic solvent is selected from a group consisting of: tetrahydrofuran (“THF”), diethyl ether, toluene, methyltertbutyl ether (“MTBE”), 2-methyl tetrahydrofuran, and mixtures thereof. Preferably, the organic solvent is MTBE.

The above solution or suspension is then combined with the reducing agent providing a mixture. Typically, the reducing agents are provided as solutions. Reducing agents include SDMA, Lithium tris[(3-ethyl-3-pentyl)oxy]aluminohydride, TBLAH or DIBALH. Preferably, the reducing agent is SDMA.

Typically, at least two equivalents of reducing agent are needed to reduce the starting compound of formula II to BIP. Preferably, about 2 to about 5 moles equivalent of reducing agent per mole equivalent of the compound of formula II are added, more preferably about 2 to about 4.2 moles equivalent of reducing agent per mole equivalent of the compound of formula II.

The reducing agent can be added to the solution or suspension, alternatively, the solution or suspension can be added to the reducing agent. Preferably, the reducing agent is added to the solution or the suspension.

The reducing agent is used in a form of a solution. Preferably, the solvent that is used is an organic solvent; more preferably, an aprotic organic solvent, selected from a group including aromatic hydrocarbons, aliphatic hydrocarbons, chlorohydrocarbons, ethers and mixtures thereof. Preferably, the aromatic hydrocarbon is toluene. Most preferably, the aliphatic hydrocarbon is heptane, cyclohexane or hexane, preferably, the chlorohydrocarbon is dichloromethane, preferably, the ether is THF. Most preferably, the solvent is toluene.

Optionally, if the reaction mixture including the reducing agent is viscous, then additional or a second organic solvent can be added to provide a more dilute reaction mixture in a form of a solution. Preferably, the second organic solvent is the same solvent used in the previous step.

Typically, the reaction between the compound of formula II and the reducing agent is exothermic. Thus, the temperature of the reaction mixture may determine the rate of addition.

Preferably, the addition is done a drop-wise fashion.

Preferably, the drop wise addition is done during a period of about 10 to about 60 minutes, more preferably, for about 10 to about 30 minutes.

Typically, the addition leads to an increase in the temperature of the reaction mixture. Thus, the temperature of the reaction mixture is kept below 70° C., preferably, the temperature is kept below 60° C., more preferably, the temperature is kept below 50° C., most preferably, the temperature is kept at about 47° C. to about 48° C.

The obtained mixture is then maintained to obtain BIP. Preferably, the mixture is stirred during this period. The mixture is maintained preferably, at a temperature of about 40° C. to about 50° C. Preferably, the mixture is maintained for a about 30 minutes to about 4 hours, more preferably, for about 30 minutes to about 3 hours, which is a significantly shorter reaction time as compared to the reaction time of the prior art. The longer reaction time of the prior art is most likely due to the use of powdery LAH, which may lose reactivity, as mentioned before, leading to an incomplete reaction. Hence, in order to compensate for the loss of reactivity, a longer reaction time is required, during which constant addition of LAH is conducted. However, although the reaction time in the process of the present invention is shorter, the conversion is high and so are the yield and the purity.

The process for preparing BIP may further comprise a recovery process. The recovery of BIP of the present invention is much simpler and more efficient than the recovery described in the prior art, especially for removing benzyl alcohol, which is a by-product of the reaction (see example 5 vs. example 9).

The recovery process comprises an extraction step, in which BIP is transformed into its acid salt, thus being separated from the organic impurities, such as benzyl alcohol, and is then converted back to BIP by addition of a base. Preferably, prior to the addition of the acid, the reaction mixture is cooled and quenched by the addition of a base, providing a two phase system, from which BIP is recovered as mentioned above. Thus, BIP can be recovered without the need to filter out the obtained aluminium hydroxide and other salts before the addition of the acid, as done in the prior art. Preferably, the cooling temperature is 15° C. to 20° C.

Preferably, the base is either sodium hydroxide or potassium hydroxide, more preferably, sodium hydroxide.

Preferably the base is added in a form of an aqueous solution.

The recovery provides crude BIP, which can be further purified by crystallizing. Preferably, the solvent for crystallization is toluene and a mixture toluene and n-heptane.

Typically, crude BIP as obtained in the prior art comprises three main impurities of the following formulas,

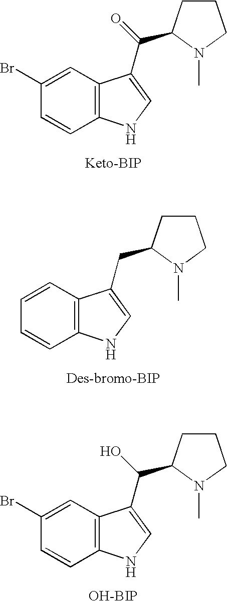

as exemplified in example 9.

These impurities are difficult to remove by conventional purification methods if they are obtained above a certain level.

In addition, if the level of these impurities is too high, BIP will not be able to crystallize, and thus will require a more complicated and time consuming purification process, such as column chromatography.

Unless mentioned otherwise, the purity measurement is by area % as measured by HPLC.

Usually, the crystallization of BIP follows a process in which crude BIP is combined with another solvent followed by filtering off a precipitate comprising of keto-BIP, providing a filtrate having BIP. The solvent can be MTBE or ethylacetate.

Typically, the first crystallization provides crystalline BIP having purity of at least 91%, and the second crystallization provides at least 96%.

The process of preparing BIP may further comprise a process of converting BIP to eletriptan and salts thereof, preferably the salt is HBr.

Having described the invention with reference to certain preferred embodiments, other embodiments will become apparent to one skilled in the art from consideration of the specification. The invention is further defined by reference to the following examples describing in detail the process for the preparation of 5-bromo-3-[(R)-1-methyl-pyrrolidin-2-ylmethyl]-1H-indole. It will be apparent to those skilled in the art that many modifications, both to materials and methods, may be practiced without departing from the scope of the invention.

### EXAMPLES

### HPLC Method

Column: LUNA C18(2) column (250×3 mm, 5 μm).

Eluent A: 10% acetonitrile, 90% water, 10 mM SDS, and 20 mM H₃PO₄ (at pH 6.0 adjusted with NaOH).

Eluent B: 80% acetonitrile, 20% water and 10 mM SDS.

Flow-rate: 0.9 ml/min

Detector: wavelength at 220 nm.

Column temperature: 35° C.

Diluent: acetonitrile.

Sample volume: 10 μL.

Equilibrium time: 10 minutes.

Gradient flow:

| Time (minute) | % Eluent A | % Eluent B |
|---|---|---|
| 0.0 | 50.0 | 50.0 |
| 15.0 | 44.0 | 56.0 |
| 35.0 | 0.0 | 100.0 |
| 37.0 | 50.0 | 50.0 |

Sample solution preparation: about 25 mg of BIP sample was accurately weighed into a 100 ml volumetric flask, sample was dissolved and adjusted to full volume with acetonitrile.

Reaction mixture or mother liquor preparation: about 30 mg of sample (several drops of sample transferred) was accurately weighed into a 100 ml volumetric flask, sample was dissolved and adjusted to full volume with acetonitrile.

Relative response factors to BIP (at 220 nm):

| Compound | RRF |
|---|---|
| Benzyl alcohol | 7.35 |
| Des-Bromo BIP | 0.63 |
| OH-BIP | 0.89 |
| Toluene | 8.70 |
| Keto-BIP | 1.14 |
| BIPCAM | 3.18 |
| BIP | 1.00 |
| Unknown | 1.00 |

Area % as used herein, unless defined otherwise, refers to the peak area of a compound as measured by HPLC.

Norm % as used herein, unless defined otherwise, refers to the normalized percent of a compound as measured by HPLC.

### Example 1

### Preparation of 5-Bromo-3-((R)-1-methyl-pyrrolidin-2-ylmethyl)-1H-indole (BIP)

A solution of (R)-2-(5-Bromo-1H-indole-3-carbonyl)-pyrrolidine-1-carboxylic acid benzyl ester (60.0 g, 1.0 eq.) in dry tetrahydrofuran (600 ml) was added dropwise over the period of 60 min to the stirred 70% solution of SDMA in toluene (210 ml, 2.1 eq.) diluted by dry tetrahydrofuran (270 ml) under atmosphere of dry nitrogen while maintaining the temperature between 30 and 40° C.

The mixture was stirred and heated to the temperature of 50° C. for 60 min. Then the mixture was cooled to 5° C. and water (100 ml) was carefully added, followed by 10% aqueous solution of sodium hydroxide (200 ml) and more water (400 ml). The resulting mixture was diluted with toluene (400 ml) and the phases were separated. The bottom aqueous phase was twice extracted with toluene (200 ml). The obtained organic phases were joined, dried over anhydrous sodium sulphate and evaporated under reduced pressure to the honey-like residue, which was chromatographed on basic alumina (500 g). Elution with toluene sequentially polarized with acetone gave a product of BIP (30.0 g, 73% yield) as a white solid (crystal from toluene/heptane).

### Example 2

### Preparation of 5-Bromo-3-((R)-1-methyl-pyrrolidin-2-ylmethyl)-1H-indole (BIP)

A solution of (R)-2-(5-Bromo-1H-indole-3-carbonyl)-pyrrolidine-1-carboxylic acid benzyl ester (52.3 g, 1.0 eq.) in dry tetrahydrofuran (250 ml) was added dropwise over the period of 40 to 50 min to the stirred 70% solution of SDMA in toluene (180 ml, 2.06 eq.) diluted by dry tetrahydrofuran (100 ml) under atmosphere of dry nitrogen in such a way the temperature would not exceed 50° C.

The resulting mixture was stirred and maintained at the temperature of 50° C. for 60 min. Then the mixture was cooled to 5° C. and the aqueous solution (5 weight per weight) of sodium hydroxide (550 ml) was carefully added while the temperature of the mixture is maintained below 25° C. The mixture was diluted with toluene (300 ml) and the phases were separated. The bottom aqueous phase was extracted with toluene (200 ml). The obtained organic phases were joined and extracted with diluted 8 w % aqueous solution of acetic acid (three times 200 ml)

The obtained acidic extracts were joined and toluene (300 ml) was added. The resulting heterogeneous mixture was vigorously stirred and cooled to 10° C. Aqueous solution (10 w %) of sodium hydroxide (250 ml) was added and the phases were separated after 10 min of stirring. The bottom aqueous phase was extracted with toluene (twice 200 ml) and then it was discarded. All toluene extracts containing the product were joined, concentrated to the crystallization volume (130 ml), diluted by n-heptane (130 ml) and let to crystallize overnight. The crystalline product was separated on a Büchner funnel, washed with n-heptane and dried under vacuum. The product (31.8 g) obtained in such a way could be directly used as a starting material for further synthetic steps or it can be purified by either re-crystallization or chromatography to obtain pure 5-Bromo-3-((R)-1-methyl-pyrrolidin-2-ylmethyl)-1H-indole.

### Example 3

### Preparation of 5-Bromo-3-((R)-1-methyl-pyrrolidin-2-ylmethyl)-1H-indole (BIP)

SDMA in toluene (70% solution, 80 ml, 280 mmol), diluted with dry diethylether (20 ml), was added to a stirred suspension of (R)-2-(5-Bromo-1H-indole-3-carbonyl)-pyrrolidine-1-carboxylic acid benzyl ester (24 g, 56.2 mmol) in dry diethylether (140 ml) under mild reflux in 20 min. The resulting yellow solution was refluxed for additional 4 hours at 39-40° C. The reaction was cooled to 15° C. and 5% aqueous NaOH (120 ml) was added dropwise while the temperature was maintained between 15° C. and 20° C. The reaction mixture was stirred vigorously for additional 60 min at 20° C. The organic layer was separated and extracted with 1M HCl (60-70 ml) to pH 3. The aqueous layer was extracted with toluene (2×30 ml). Then the aqueous layer was combined with toluene (100 ml) in a separatory funnel and 2M NaOH was added in portions to pH 12 (30-35 ml) and the product was extracted to the organic phase. Organic phase was separated, the aqueous phase was re-extracted with a fresh portion of toluene (50 ml) and combined toluene extracts were evaporated under reduced pressure. The residue was dissolved in toluene (30 ml) and the product was crystallized upon cooling. The product was filtered off, washed with cold toluene and cold n-heptane and dried (11.3 g; 69% yield). BIP Purity: 94.8 area % as measured by HPLC. Level of impurities found (area %): Des-Bromo-BIP: 0.96%; OH-BIP: 0.18%; Keto-BIP: 3.12%.

### Example 4

### Preparation of 5-Bromo-3-((R)-1-methyl-pyrrolidin-2-ylmethyl)-1H-indole (BIP)

SDMA in toluene (70% solution, 172 ml, 0.6 mol), diluted with dry toluene (30 ml), was added to a stirred suspension of (R)-2-(5-Bromo-1H-indole-3-carbonyl)-pyrrolidine-1-carboxylic acid benzyl ester (60 g, 0.14 mol) in dry toluene (350 ml) at 30-40° C. during 10 min. The reaction temperature was raised to 48° C. and the resulting yellow solution was stirred for 2.5 h. The reaction was cooled to 15° C. and 5% aqueous NaOH (300 ml) was added dropwise while the temperature was maintained between 15-20° C. The reaction mixture was stirred vigorously for additional 60 min at 20° C. The organic layer was separated and analyzed by HPLC. Crude BIP purity: 66.49 Norm %. Levels of impurities found (Norm %): Benzyl-alcohol: 24.11%; Des-Bromo-BIP: 1.91%; OH-BIP: 1.38%; Keto-BIP: 1.90%.

The organic layer was extracted with 2M acetic acid (1×250 ml) and (1×50 ml). The aqueous layer was combined with toluene (250 ml) in a separatory funnel and 10% NaOH was added in portions to pH 12 (250 ml) and the product was extracted to the organic phase. The organic phase was separated, the aqueous phase was re-extracted with a fresh portion of toluene (50 ml) and combined toluene extracts were evaporated under reduced pressure. The residue was dissolved in toluene (75 ml) and the product was crystallized upon cooling. The product was filtered off, washed with cold toluene and cold heptane and dried (23.5 g; 57%). BIP purity: 96.5 Norm %. Levels of impurities found (Norm %): Des-Bromo-BIP: 0.85%; OH-BIP: 0.56%; Keto-BIP: 1.90%.

### Example 5

### Preparation of 5-Bromo-3-((R)-1-methyl-pyrrolidin-2-ylmethyl)-1H-indole (BIP)

SDMA in toluene (70% solution, 40 ml, 131.2 mmol), diluted with dry toluene (5 ml), was added to a stirred suspension of (R)-2-(5-Bromo-1H-indole-3-carbonyl)pyrrolidine-1-carboxylic acid benzyl ester (12 g, 28.1 mmol) in dry toluene (70 ml) at 30-40° C. during 10 min. The temperature was raised to 48° C. and the resulting yellow solution was stirred for 2.5 h. The reaction was cooled to 15° C. and 5% aqueous NaOH (60 ml) was added dropwise while the temperature was maintained between 15-20° C. The reaction mixture was stirred vigorously for additional 60 min at 20° C. Crude BIP purity: 72.36 Norm %. Levels of impurities found (Norm %): Benzyl alcohol: 21.50%; Des-Bromo-BIP: 2.16%; OH-BIP: 0.47%; Keto-BIP: 2.14%.

### Example 6

### Preparation of 5-Bromo-3-((R)-1-methyl-pyrrolidin-2-ylmethyl)-1H-indole (BIP)

SDMA in toluene (70% solution, 72 ml, 252 mmol), diluted with dry MTBE (12 ml), was added to a stirred suspension of (R)-2-(5-Bromo-1H-indole-3-carbonyl)-pyrrolidine-1-carboxylic acid benzyl ester (24 g, 56.2 mmol) in dry MTBE (140 ml) at 30-40° C. during 10 min. The reaction temperature was raised to 48° C. and the resulting yellow solution was stirred for 3 h. The reaction was cooled to 15° C. and 5% aqueous NaOH (120 ml) was added dropwise while the temperature was maintained between 15-20° C. The reaction mixture was stirred vigorously for additional 30 min at 20° C. Organic layer was separated and extracted with 1M HCl (80 ml) to pH 3. The aqueous layer was extracted with MTBE (2×50 ml). The aqueous layer was combined with MTBE (120 ml) in a separatory funnel and 2M NaOH was added in portions to pH 12 (40-45 ml) and the product was extracted to the organic phase. The organic phase was separated, the aqueous phase was re-extracted with a fresh portion of MTBE (50 ml) and combined MTBE extracts were evaporated under reduced pressure. The residue was dissolved in toluene (30 ml) and the product was crystallized upon cooling. The product was filtered off, washed with cold toluene and cold heptane and dried (12.6 g; 77%). BIP purity: 93.7 Norm %. Level of impurities found (Norm %): Des-Bromo-BIP: 0.41%; OH-BIP: 0.55%; Keto-BIP: 4.75%.

### Example 7

### Preparation of 5-Bromo-3-((R)-1-methyl-pyrrolidin-2-ylmethyl)-1H-indole (BIP)

SDMA in toluene (70% solution, 72 ml, 252 mmol), diluted with dry 2-methyltetrahydrofurane (12 ml), was added to a stirred suspension of (R)-2-(5-Bromo-1H-indole-3-carbonyl)-pyrrolidine-1-carboxylic acid benzyl ester (24 g, 56.2 mmol) in dry 2-methyltetrahydrofurane (140 ml) at 30-40° C. during 10 min. The reaction temperature was raised to 48° C. and the resulting yellow solution was stirred for 3 h. The reaction was cooled to 15° C. and 5% aqueous NaOH (120 ml) was added dropwise while the temperature was maintained between 15-20° C. The reaction mixture was stirred vigorously for additional 30 min at 20° C. Organic layer was separated and extracted with 1M HCl (70-80 ml) to pH 3. The aqueous layer was extracted with toluene (2×50 ml). The aqueous layer was combined with toluene (120 ml) in a separatory funnel and 2M NaOH was added in portions to pH 12 (40-45 ml) and the product was extracted to the organic phase. The organic phase was separated, the aqueous phase was re-extracted with a fresh portion of toluene (50 ml) and combined toluene extracts were evaporated under reduced pressure. The residue was dissolved in toluene (30 ml) and the product was crystallized upon cooling. The product was filtered off, washed with cold toluene and cold heptane and dried (11.5 g; 70%).

### Example 8

### Preparation of 5-Bromo-3-((R)-1-methyl-pyrrolidin-2-ylmethyl)-1H-indole (BIP)

SDMA in toluene (70% solution, 143.9 ml, 504 mmol), diluted with dry THF (25 ml), was added to a stirred solution of (R)-2-(5-Bromo-1H-indole-3-carbonyl)-pyrrolidine-1-carboxylic acid benzyl ester (48 g, 112.3 mmol) in dry THF (200 ml) at 30-40° C. during 15 min. The reaction temperature was raised to 48° C. and the resulting yellow solution was stirred for 2.5 h. The reaction was diluted with toluene (200 ml) and cooled to 15° C. 5% Aqueous NaOH (240 ml) was added dropwise while the temperature was maintained between 15-20° C. The reaction mixture was stirred vigorously for additional 30 min at 20° C. The organic layer was separated, the aqueous phase was extracted with toluene (1×100 ml), both organic phases were combined and extracted with 1M HCl (160-180 ml) to pH 3. The aqueous layer was extracted with toluene (2×100 ml). The aqueous layer was combined with toluene (200 ml) in a separatory funnel and 2M NaOH was added in portions to pH 12 (80-90 ml) and the product was extracted to the organic phase. Organic phase was separated, the aqueous phase was re-extracted with a fresh portion of toluene (2×100 ml) and combined toluene extracts were evaporated under reduced pressure. The residue was dissolved in toluene (60 ml) and the product was crystallized upon cooling. The product was filtered off, washed with cold toluene (40 ml) and cold heptane (40 ml) and dried (23 g, 70%). BIP purity (Norm %): 93.0%. Level of impurities found (Norm %): Des-Bromo-BIP: 0.70%; OH-BIP: 0.07%; Keto-BIP: 5.46%.

### Example 9

### Preparation of 5-Bromo-3-((R)-1-methyl-pyrrolidin-2-ylmethyl)-1H-indole (BIP) according to U.S. Pat. No. 5,545,644, example 27

| BIPCAM | 3.64 g (8.52 mmol) |
|---|---|
| LAH | 1.26 g (33.2 mmol, 3.9 eq) |
| THF | 122.5 |

A solution of (R)-2-(5-Bromo-1H-indole-3-carbonyl)-pyrrolidine-1-carboxylic acid benzyl ester (BIPCAM) (3.64 g, 8.55 mmol) in dry THF (70 ml) was added drop-wise to a stirred suspension of lithium aluminium hydride (0.945 g, 24.9 mmol) in dry THF (52.5 ml) at room temperature under an atmosphere of dry nitrogen. The mixture was heated under reflux with stirring for 18 h and then cooled. Additional lithium aluminium hydride (175 mg, 4.61 mmol) was added and refluxing was continued for an additional 3 h. The mixture was again cooled, lithium aluminium hydride (140 mg, 3.69 mmol) was added, and refluxing was continued for a further 18 h. The mixture was cooled and H₂O (1.54 ml) was carefully added with stirring, followed by 20% aqueous NaOH (1.54 ml), followed by more H₂O (4.66 ml). The mixture was stirred for 30 min, then diluted with ethyl acetate (50 ml) and filtered through Celite. The filtrate was washed with H₂O (50 ml), brine (50 ml) and then dried with Na₂SO₄. Evaporation of the solvent gave an oil (3.6 g) which was chromatographed on silica gel, eluted with dichloromethane/ethanol/conc. aqueous ammonia (90:10:0.5) to obtain the title compound (1.18 g, 47%) as a light yellow oil

The product precipitated from dichloromethane/hexane (920 mg, 37%).

### HPLC Analysis:

Crude reaction mixture (Norm %):

| | |
|---|---|
| benzyl alcohol | 26.25% |
| des-Bromo-BIP | 1.03% |
| OH-BIP | 10.75% |
| keto-BIP | 0.14% |
| BIP | 57.02% |

Purified product (Norm %):

| | |
|---|---|
| des-Bromo-BIP | 0.52% |
| OH-BIP | 0.14% |
| keto-BIP | 0.11% |
| BIP | 97.8% |

### Example 10

### Final purification of 5-Bromo-3-((R)-1-methyl-pyrrolidin-2-ylmethyl)-1H-indole (BIP)

Crude 5-Bromo-3-((R)-1-methyl-pyrrolidin-2-ylmethyl)-1H-indole (20 g) was stirred with MTBE (100 ml) at 20° C. for 1 h. A white solid was filtered off and washed with MTBE (20 ml). The filtrate was evaporated to dryness and the residue was crystallized from toluene (30 ml). The product was filtered off washed with cold toluene and heptane and dried (yield: 17.1 g, 86%). BIP purity: 97.7 Norm %. Level of impurities found (Norm %): Des-Bromo-BIP: 0.60%; OH-BIP: 0.30%; Keto-BIP: 1.30%.

### Example 11

### Final purification of 5-Bromo-3-((R)-1-methyl-pyrrolidin-2-ylmethyl)-1H-indole (BIP)

Crude 5-Bromo-3-((R)-1-methyl-pyrrolidin-2-ylmethyl)-1H-indole (20 g) was dissolved in hot ethyl acetate (100 ml) the solution was cooled to 20° C. and stirred for 2 h. A white solid was filtered off and washed with ethyl acetate (20 ml). The filtrate was evaporated to dryness and the residue was crystallized from toluene (30 ml). The product was filtered off washed with cold toluene and heptane and dried (yield: 16.6 g, 83%). BIP purity 97.9 Norm %. Level of impurities found (Norm %): Des-Bromo-BIP: 0.21%; OH-BIP: 0.28%; Keto-BIP: 1.48%.

### Example 12

### Preparation of (R)-2-(5-bromo-1H-indole-3-carbonyl)-pyrrolidine-1-carboxylic acid benzyl ester of formula II, according to U.S. Pat. No. 5,545,644, example 56

Two solutions containing the reactants were prepared separately as follows.

To a stirred solution of N-benzyloxycarbonyl-D-proline (291.93 g) in dichloromethane (291.9 ml) and toluene (370.8 ml) containing N,N-dimethylyformamide (1.46 ml) was added oxalyl chloride (102.2 ml) in toluene (291.9 ml) and the resulting solution was stirred at ambient temperature overnight. The solution was then purged by passing a stream of dry nitrogen gas for five hours. This solution of N-benzyloxycarbonyl-D-proline acid chloride was ready for use.

In parallel, a solution of ethyl magnesium bromide (800 ml of a 3M solution in ether) was added dropwise over one hour to a stirred solution of 5-bromoindole (459.15 g) in dichloromethane (4391.4 ml). The mixture was stirred and heated at reflux for 30 minutes then cooled to −20° C. The above solution of N-benzyloxycarbonyl-D-proline acid chloride was added dropwise with stirring (over one hour) and stirring was continued for a further 30 minutes. Then a solution of ammonium chloride (1122.3 g) in water (5855.3 ml) was added at this temperature and the mixture allowed to warm to room temperature. Further ammonium chloride (1452.3 g) in water (2000 ml) was added to allow separation of the phases. The phases were separated and the aqueous phase extracted with dichloromethane (1.95 L) then discarded. The combined organic phases were washed with aqueous sodium bicarbonate solution (2.7 L), then with brine (1 L) before concentration to low volume (about 1 L). This concentrate was diluted with ethyl acetate (1250 ml) and then further diluted with hexane (1250 ml). The resulting slurry was stirred at ambient temperature before collection of the title compound (361.4 g) by filtration (washing with 1:1 ethyl acetate:hexane-2×300 ml) and drying in vacuo: LRMS m/z (relative intensity) 428 (M⁺ with ⁸¹Br, 5) 426 (M⁺ with ⁷⁹Br, 5), 224 (19), 222 (21), 204 (62), 160 (68), 91 (100). Anal. Calcd. for C₂₁H₁BrN₂O₃: C, 59.02; H, 4.48; N, 6.56. Found: C, 58.85; H, 4.51; N, 6.38.

1. A process for preparing 5-bromo-3-[(R)-1-methyl-pyrrolidin-2-ylmethyl]-1H-indole 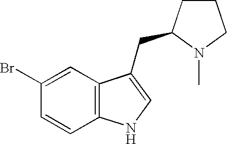
comprising: reacting (R)-2-(5-bromo-1H-indole-3-carbonyl)-pyrrolidine-1-carboxylic acid benzyl ester of Formula II 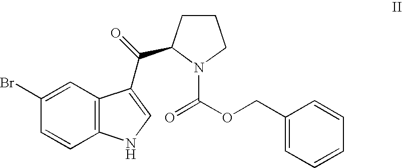
with a reducing agent selected from the group consisting of: sodium dihydro-bis(2-methoxyethoxy)aluminate, lithium tris[(3-ethyl-3-pentyl)oxy]aluminohydride, lithium tri-tert-butoxyaluminum hydride and diisobutylaluminum hydride to yield 5-bromo-3-[(R)-1-methyl-pyrrolidin-2-ylmethyl]-1H-indole.

2. The process according to claim 1, wherein reacting (R)-2-(5-bromo-1H-indole-3-carbonyl)-pyrrolidine-1-carboxylic acid benzyl ester of Formula II with the reducing agent is done in an aprotic organic solvent.

3. The process according to claim 2, wherein the aprotic organic solvent is selected from the group consisting of tetrahydrofuran, diethyl ether, toluene, methyltertbutyl ether, 2-methyl tetrahydrofuran, and mixtures thereof.

4. The process according to claim 3, wherein the aprotic organic solvent is methyltertbutyl ether.

5. The process according to any of claims 1 to 4, wherein the reducing agent is present in about 2 to about 5 moles equivalent per mole equivalent of (R)-2-(5-bromo-1H-indole-3-carbonyl)-pyrrolidine-1-carboxylic acid benzyl ester.

6. The process according to claim 1, wherein the reaction mixture is a solution.

7. The process according to claims 1, wherein the reaction is performed at a temperature below 50° C.

8. The process of claim 1, further comprising recovering 5-bromo-3-[(R)-1-methyl-pyrrolidin-2-ylmethyl]-1H-indole.

9. The process according to claim 8, further comprising a quenching step by adding a base to the reaction mixture to provide a two phase system.

10. The process according to claim 8, further comprising an extraction step by extracting the 5-bromo-3-[(R)-1-methyl-pyrrolidin-2-ylmethyl]-1H-indole and removing the solvent.

11. The process according to any of the claims 1 or 8 further comprising crystallizing the 5-bromo-3-[(R)-1-methyl-pyrrolidin-2-ylmethyl]-1H-indole from a crystallization solvent.

12. The process according to claim 11, wherein the crystallization solvent is toluene, or mixtures of toluene and n-heptane.

13. The process according to claim 11, wherein the crystallization solvent is toluene.

14. A process for preparing eletriptan and salts thereof comprising preparing 5-bromo-3-[(R)-1-methyl-pyrrolidin-2-ylmethyl]-1H-indole by a process according to claim 1, and converting the obtained 5-bromo-3-[(R)-1-methyl-pyrrolidin-2-ylmethyl]-1H-indole to eletriptan and salts thereof.

15. The process according to claim 14, wherein the eletriptan salt is eletriptan hydrobromide.

16. The process for preparing 5-bromo-3-[(R)-1-methyl-pyrrolidin-2-ylmethyl]-1H-indole according to claim 1, wherein the 5-bromo-3-[(R)-1-methyl-pyrrolidin-2-ylmethyl]-1H-indole having a purity of at least 91% as analyzed by HPLC.

17. The process for preparing 5-bromo-3-[(R)-1-methyl-pyrrolidin-2-ylmethyl]-1H-indole according to claim 1, wherein the 5-bromo-3-[(R)-1-methyl-pyrrolidin-2-ylmethyl]-1H-indole having a purity of at least 96% as analyzed by HPLC.

18. The process for preparing 5-bromo-3-[(R)-1-methyl-pyrrolidin-2-ylmethyl]-1H-indole according to claim 16, wherein the HPLC conditions include a C18(2) column (250×3 mm, 5 μm) column, eluent A of 10% acetonitrile, 90% water, 10 mM SDS, and 20 mM H₃PO₄ (at pH 6.0 adjusted with NaOH); eluent B: 80% acetonitrile, 20% water and 10 mM SDS, and the UV detector is at a wavelength of 220 nm.
